(19) United States
(12) Patent Application Publication
(10) Pub. No.: US 2015/0185241 A1
(43) Pub. Date: Jul. 2, 2015

(54) Tilidine Immunodetection

(71) Applicant: Randox Laboratories Limited, Crumlin (GB)

(72) Inventors: Elouard Benchikh, Crumlin (GB); Peter Stephen Fitzgerald, Crumlin (GB); Ivan McConnell, Crumlin (GB); Philip Lowry, Crumlin (GB)

(21) Appl. No.: 14/576,618

(22) Filed: Dec. 19, 2014

(30) Foreign Application Priority Data
Dec. 23, 2013 (GB) 1322935

(51) Int. Cl. G01N 33/94; C07C 229/46; C07K 16/44

(52) U.S. Cl. CPC G01N 33/9486; C07K 16/44; C07C 229/46; C07C 2101/14; C07C 2101/16; G01N 2430/00

(57) ABSTRACT

An immunoassay for the detection of tilidine and nortilidine is described. The invention also describes antibodies and kits.

### BACKGROUND

Tilidine, systematic name (±)-trans-ethyl 2-(N,N-dimethylamino)-1-phenylcyclohex-3-enylcarboxylate, is a synthetic opioid analgesic, commonly prescribed for acute and chronic pain. Tilidine is a mixture of the following two trans isomers:

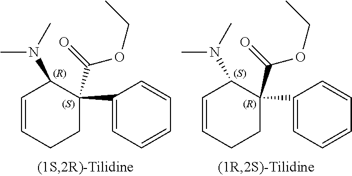

Tilidine is depicted hereunder, for convenience, as a single trans isomer—the 1S, 2R trans isomer. The depicted 1S, 2R trans isomer is intended to embrace both trans isomers, being the 1R, 2S and the 1S, 2R trans isomers. Upon ingestion, tilidine is rapidly metabolised, undergoing demethylation to the pharmacologically active nortilidine, systematic name (±)-trans-ethyl 2-(N-methylamino)-1-phenylcyclohex-3-enylcarboxylate; the minor metabolite (±)-trans-ethyl 2-amino-1-phenylcyclohex-3-enylcarboxylate, trivial name bisnortilidine is also formed (FIG. 1 illustrates the 1S, 2R trans isomer, for convenience, rather than both the 1R, 2S and the 1S, 2R trans isomers, for each of tilidine, nortilidine and bisnortilidine). The opioid activity of tilidine has led to its abuse and there is a need for its detection for toxicological purposes e.g. in individuals who drive while intoxicated, and for therapeutic drug monitoring. Various methods of detecting tilidine and its metabolites, mostly based on expensive, relatively complex laboratory-based mass-spectrometry and chromatographic techniques, are described e.g. Kohler et al (Anal. Bioanal. Chem., 2011, 400:17-23) describe a liquid chromatography-mass-spectrometry (LC-MS-MS) system. However, more sensitive, cheaper and simpler detection methods amenable to application in the field are desirable.

### FIGURES

FIG. 1 Structure of the 1S, 2R trans isomer of each of tilidine, nortilidine and bisnortilidine

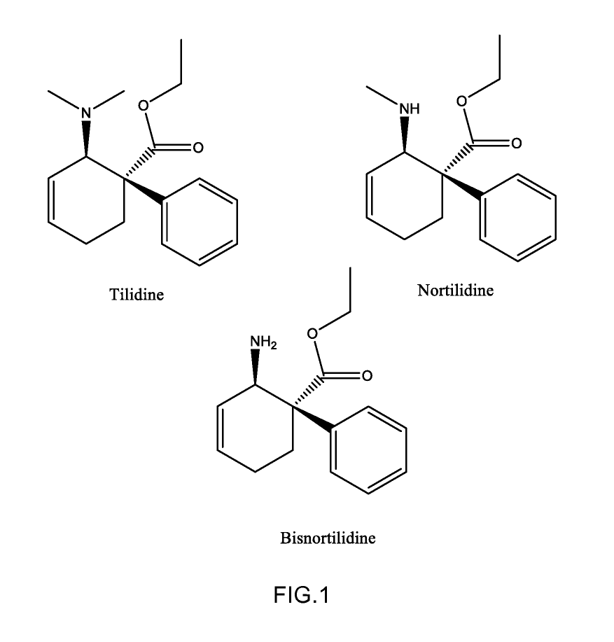

FIG. 2 Preparation of hapten N-(carboxypropyl)nortilidine (the structures that are illustrated are those of the 1S, 2R trans isomer, although it is intended that both trans isomers (the 1R, 2S and the 1S, 2R isomers) are to be embraced by the illustrated preparative method)

### DESCRIPTION OF THE INVENTION

The invention describes a method of detecting or determining tilidine and nortilidine in an in vitro sample of an individual comprising; contacting the sample with one or more detecting agents and one or more antibodies; detecting, or determining the quantity of the one or more detecting agents; and deducing from calibrators the presence of or amount of tilidine and nortilidine in the sample, the one or more antibodies characterised by having been derived from a trans 1S, 2R immunogen of formula I

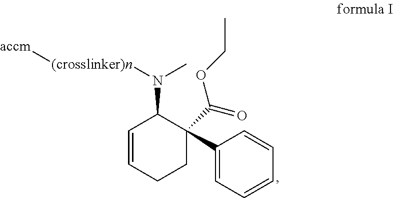

the 1R, 2S trans isomer thereof, or a mixture thereof, preferably a racemic mixture
wherein n=0 or 1; and, when n=1, the crosslinker joins the nitrogen atom to the accm, the antigenicity conferring carrier material. Optionally, the immunogen is derived from a mixture, preferably a racemic mixture, of the 1S, 2R trans and the 1R, 2S trans isomers. By ‘detecting’ is meant qualitatively analysing for the presence or absence of a substance. By ‘determining’ is meant quantitatively analysing for the amount of substance present. As the antibodies of the invention are able to bind tilidine and nortilidine and, optionally, bisnortilidine, quantitative analysis will take the form of measuring the calibrator-equivalent amount. Any suitable in vitro biological sample may be used, but blood and urine are preferred.

The invention further describes an immunogen of formula I in which the crosslinker, if present, is, optionally, —X—Y—. Although any suitable crosslinker may be used (see Methods and Results section), a preferred crosslinker is represented by —X—Y— in which X, which is attached to the nitrogen atom, is selected from a group comprising a substituted or unsubstituted, straight or branched chain, saturated or unsaturated alkylene moiety; preferably X is C1-C10 alkylene, more preferably a C1-C6 alkylene moiety; still more preferably a C3 alkylene moiety.

Y is a divalent functional group selected from —C(O)—, —NH—, maleimido

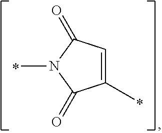

—N—C(O)—, —N—C(S)—, —S—, —S(O)₂—, and —S(O)— wherein the bracketed atom is attached to the preceding C-atom or S-atom by a double bond e.g. —C(O)— refers to carbonyl. In a preferred embodiment, the crosslinker is —CH₂—CH₂—CH₂—C(O)—. The accm is a standard component of an immunogen derived from a small molecule and can be any macromolecule that confers immunogenicity to the non-immunogenic small molecule (see Methods and Results section) but is preferably chosen from keyhole limpet haemocyanin (KLH), bovine thyroglobulin (BTG), bovine serum albumin (BSA), egg ovalbumin, bovine gamma globulin or cationised BSA.

The invention further describes an antibody derived from a trans 1S, 2R immunogen of formula I or the 1R, 2S trans isomer thereof. Optionally, the antibody is derived from a mixture, preferably a racemic mixture, of the 1S, 2R trans and the 1R, 2S trans isomers. The antibody is further characterised in being specific to an epitope of tilidine. By specific it is meant that the antibody preferably binds or has greater affinity for tilidine compared to all other molecules. The relative degree of binding of an antibody to a molecule can be described by its cross-reactivity (CR) which can be derived from a suitable metric such as the IC₅₀ (see Tables 1 and 2), the molecule with the greatest amount of binding being given a CR value of 100%. Thus, the antibody of the current invention has a CR of 100% to tilidine. The antibody preferably is cross-reactive to nortilidine. The extent of cross-reactivity of the antibody to nortilidine is preferably >10%, more preferably >15%, still more preferably >20%. Preferably, the antibody also shows cross-reactivity to bisnortilidine. The extent of cross-reactivity of the antibody to bisnortilidine is preferably >7.5%, more preferably >10%, more preferably >12.5%.

In a further embodiment, the invention describes a kit for detecting or determining tilidine and nortilidine; the kit comprises at least one antibody specific to tilidine and having cross-reactivity to nortilidine. The kit may also contain a detecting agent comprising a hapten based on the tilidine structure. The detecting agent comprises a suitable hapten, preferably the haptens disclosed herein, covalently bonded to a detectable labelling agent, the hapten moiety being able to bind to the antibodies of the invention. Preferably, the labelling agent is selected from an enzyme, a luminescent substance, a radioactive substance, or a mixture thereof. More preferably, the labelling agent is an enzyme, preferably a peroxidase, most preferably horseradish peroxidise (HRP). The kit may comprise one or more antibodies of the invention and one or more additional antibodies with different molecular specificities i.e. these additional antibodies do not bind to the same structural epitopes as the antibodies of the invention. Such an arrangement enables a multiplexing approach to the detection or determination of drugs of abuse. The multiplexing approach preferably makes use of a planar substrate to which the antibodies are attached, such as a ceramic chip or an appropriately surface-modified glass slide. Beads may also be used.

### Methods and Results

### Preparation of Haptens, Immunogens and Detecting Agents

The general process of immunogen formation is well known. Briefly, a hapten (a pre-immunogenic structure), whose structure incorporates the structural epitope(s) to be recognized by the antibody, is synthesized by introducing a crosslinker. The crosslinker provides the functional groups which will enable an antigenicity conferring carrier material (accm) to be attached to the hapten. The crosslinker is not always necessary if it is considered that the accm can be directly attached to an existing molecule which incorporates the required structural elements for immunogen attachment. The crosslinker may also be transient in nature, being a structural group which activates the hapten to accm introduction but which is not present in the final immunogen (e.g. N,N-dicyclohexylcarbodiimide and N-hydroxysuccinimide). Further examples of crosslinking agents include 1-cyclohexyl-3-(2-morpholino-yl-ethyl)carbodiimide, 1-ethyl-3-(3-dimethylaminopropyl)carbodiimide (EDC), 2-iminothiolane, 3-maleimidobenzoic acid, glutardialdehyde, succinimidyl 4-(N-maleimidomethyl)cyclohexane-1-carboxylate, N-succinimidyl-3-(2-pyridyldithio) propionate. A comprehensive list of crosslinkers can be found in the Thermo Scientific Pierce Crosslinking Technical Handbook, 2009. As previously described, although small molecules (haptens) provide defined structural epitopes, they are not in themselves immunogenic and therefore need to be conjugated to carrier materials, which will elicit an immunogenic response when administered to a host animal. Appropriate carrier materials commonly contain poly(amino acid) segments and include polypeptides, proteins and protein fragments. Illustrative examples of useful carrier materials are BSA, egg ovalbumin, bovine gamma globulin, cationized serum albumin, BTG, KLH, dextrans etc. Alternatively, synthetic poly(amino acids) having a sufficient number of available amino groups, such as lysine, may be employed, as may other synthetic or natural polymeric materials bearing reactive functional groups. Also, carbohydrates, yeasts or polysaccharides may be conjugated to the hapten to produce an immunogen (Bioconjugate Techniques, G. Hermanson, ed, Academic Press, 1996, 785 pp, lists common carrier proteins). The haptens can also be coupled to a detectable labelling agent such as an enzyme (for example, horseradish peroxidase), a substance having fluorescent properties or a radioactive label for the preparation of detecting agents for use in the immunoassays. The fluorescent substance may be, for example, a monovalent residue of fluorescein or a derivative thereof. Immunogen formation for the invention described herein involves conventional conjugation chemistry. In order to confirm that adequate conjugation of hapten to carrier material has been achieved, prior to immunisation, each immunogen is evaluated using matrix-assisted UV laser desorption/ionisation time-of-flight mass spectroscopy (MALDI-TOF MS). Formation of the conjugate (detecting agent) from a hapten follows similar principles to immunogen formation.

### General Procedure for MALDI-TOF Analysis of Immunogens.

MALDI-TOF mass spectrometry was performed using a Voyager STR Biospectrometry Research Station laser-desorption mass spectrometer coupled with delayed extraction. An aliquot of each sample to be analysed was diluted in 0.1% aqueous trifluoroacetic acid (TFA) to create 1 mg/ml sample solutions. Aliquots (1 μl) were analysed using a matrix of sinapinic acid and bovine serum albumin (Fluke) was used as an external calibrant.

### Preparation of Antisera

In order to generate polyclonal antisera, an immunogen of the present invention is mixed with Freund's adjuvant and the mixture is injected into a host animal, such as rabbit, sheep, mouse, guinea pig or horse. Sheep are the preferred host animal. Further injections (boosts) are made and serum is sampled for evaluation of the antibody titre. When the optimal titre has been attained, the host animal is bled to yield a suitable volume of specific antiserum. The degree of antibody purification required depends on the intended application. For many purposes, there is no requirement for purification, however, in other cases, such as where the antibody is to be immobilised on a solid support, purification steps can be taken to remove undesired material and eliminate non-specific binding.

### Immunoassay Development

The process of developing an immunoassay is well known to the person skilled in the art. Briefly, for a competitive immunoassay in which the target analyte is a non-immunogenic molecule such as a hapten, the following process is conducted: antibodies are produced by immunising an animal, preferably a mammalian animal, by repeated administration of an immunogen of the present invention. The serum from the immunised animal is collected when the antibody titre is sufficiently high. A detecting agent is added to a sample containing the target analyte and the raised antibodies, and the detecting agent and analyte compete for binding to the antibodies. The process may comprise fixing said serum antibodies to a backing substrate such as a microtitre plate, a polystyrene solid support or a ceramic chip. The antibodies can be polyclonal or monoclonal. The signal emitted in the immunoassay is proportionate to the amount of detecting agent bound to the antibodies which in turn is inversely proportionate to the analyte concentration. The signal can be detected or quantified by comparison with a calibrator.

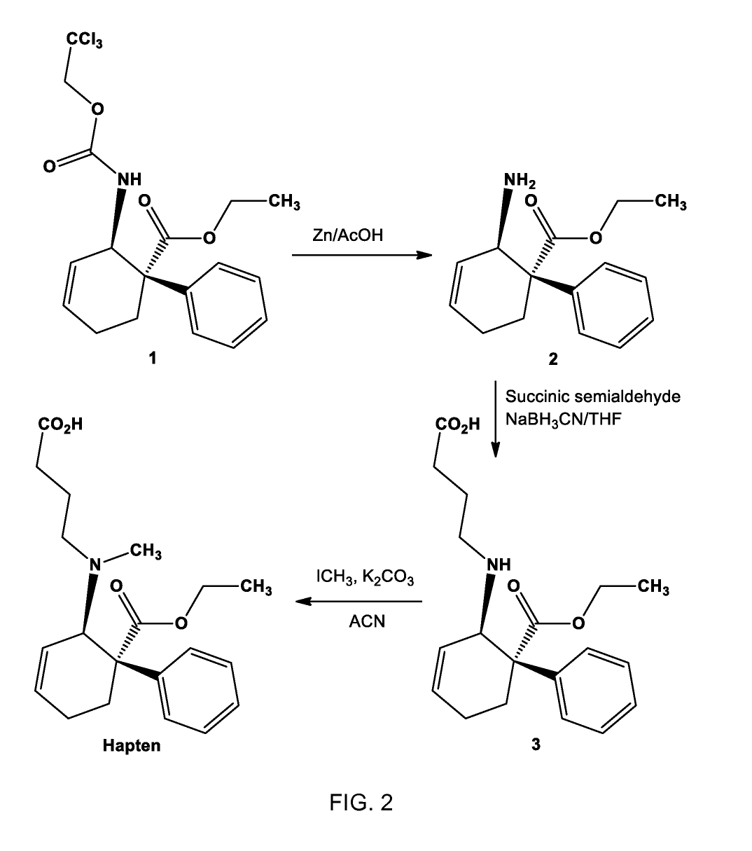

In the following Examples, the numbers following chemical names refer to the structure of FIG. 2.

### Example 1

### Preparation of Bisnortilidine 2

Zinc activation: Zn (5 g) was washed successively with HCl (2%), water, ethanol and finally with ether before being dried under high vacuum. To a solution of (±)-trans N-(2,2,2-trichloroethoxy)carbonyl bisnortilidine 1 (900 mg, 2.14 mmol; Toronto Research Chemicals Inc.) in acetic acid (AcOH) (10.8 ml) at room temperature was added Zinc dust activated (1.43 g, 0.021 mol) portion-wise and the resulting mixture was stirred at room temperature overnight. The reaction mixture was filtered and washed by acetic acid. The combined acetic acid was removed in vacuo and the resulting residue was taken into NaOH (2N) solution and extracted several times with chloroform. The organic layer were dried over sodium sulfate, filtered and concentrated to dryness to give 593.5 mg of bisnortilidine 2 as clear oil.

### Example 2

### Preparation of N-(carboxypropyl)bisnortilidine 3

To a cooled solution 0° C. of bisnortilidine 2 (469 mg, 1.91 mmol) in anhydrous tetrahydrofuran (THF) (3 ml) was added aqueous succinic semialdehyde (1.69 ml, 2.48 mmol) followed by the addition of sodium cyanoborohydride (120 mg, 1.19 mmol). The reaction mixture was stirred for 2 hours. To this solution was added a solution of HCl (1N) (3 ml) and the reaction mixture stirred at room temperature for 2 hours. The reaction mixture was concentrated in vacuo and the residue made neutral with a solution of NaOH (1N) and extracted with chloroform. The aqueous phase was concentrated in vacuo and extracted with (10%) methanol in chloroform. The organic fractions were combined and evaporated to give 331.7 mg of N-(carboxypropyl)bisnortilidine 3.

### Example 3

### Preparation of N-(carboxypropyl)nortilidine (Hapten)

To solution of N-(carboxypropyl)bisnortilidine 3 (331.7 mg, 1 mmol) in acetonitrile (ACN) (2 ml) was added potassium carbonate (317 mg, 2.3 mmol) and methyl iodide (93.4 μl, 1.5 mmol) and the reaction mixture was stirred at room temperature overnight. The solution was concentrated in vacuo and the residue was taken in water. The mixture was neutralized to pH7 with HCl (1N) and extracted with ethyl acetate. The combined organic layers were dried over sodium sulfate, filtered and concentrated to dryness. The residue was purified by column chromatography (silica gel, 10% methanol in chloroform) to give 146 mg of N-(carboxypropyl)nortilidine (Hapten) as oil. The hapten is a racemic mixture of the 1S, 2R trans and the 1R, 2S trans isomers.

### Example 4

### Conjugation of N-(carboxypropyl)nortilidine (Hapten) to BSA (Immunogen-I)

To a solution of N-(carboxypropyl)nortilidine (Hapten) (38.7 mg, 0.112 mmol) in DMF (1.0 ml) was added N,N-dicyclohexylcarbodiimide (DCC) (25.35 mg, 0.123 mmol) and N-hydroxysuccinimide (14.13 mg, 0.123 mmol) and the mixture stirred at room temperature overnight. The dicyclohexylurea formed was removed by filtration and the solution was added dropwise to a solution of BSA (150 mg, 2.3 mmol) in 50 mM sodium bicarbonate solution (pH 8.5) (10 ml). The mixture was then stirred overnight at 4° C. The solution was then dialysed against 50 mM phosphate buffer pH 7.2 (3 changes) for 24 hours at 4° C., and freeze-dried. MALDI results showed 24.99 molecule of N-(carboxypropyl)nortilidine (Hapten) had been conjugated to one molecule of BSA.

### Example 5

### Conjugation of N-(carboxypropyl)nortilidine (Hapten) to bovine thyroglobulin (BTG) (Immunogen-II)

To a solution of N-(carboxypropyl)nortilidine (Hapten) (46.63 mg, 0.135 mmol) in DMF (1.0 ml) was added N,N-dicyclohexylcarbodiimide (DCC) (30.7 mg, 0.149 mmol) and N-hydroxysuccinimide (17.13 mg, 0.149 mmol) and the mixture stirred at room temperature overnight. The dicyclohexylurea formed was removed by filtration and the solution was added dropwise to a solution of BTG (150 mg, 2.25 umol) in 50 mM sodium bicarbonate solution (pH 8.5) (10 ml). The mixture was then stirred overnight at 4° C. The solution was then dialysed against 50 mM phosphate buffer pH 7.2 (3 changes) for 24 hours at 4° C., and freeze-dried.

### Example 6

### Conjugation of N-(carboxypropyl)nortilidine (Hapten) to horseradish peroxidase (HRP)

EDC hydrochloride (10 mg) was dissolved in water (0.5 ml) and immediately added to a solution of N-(carboxypropyl)nortilidine (Hapten) (2 mg) in DMF (0.2 ml). After mixing, this solution was added dropwise to a solution of HRP (20 mg) in water (1 ml). Sulfo-NHS (5 mg) was added and the reaction mixture was incubated in the dark at room temperature overnight. Excess hapten was removed with double PD-10 columns (Pharmacia) in series, pre-equilibrated with PBS at pH 7.2. The hapten-HRP conjugate was then dialysed overnight against 10 L of PBS at pH 7.2 at 4° C.

### Example 7

### Immunoassay Development

The wells of an enhanced binding 96 well polystyrene microtitre plate were coated with the Ig fraction of the antiserum raised to Immunogen II (N-(carboxypropyl)nortilidine-BTG—example 5), diluted in 10 mMTris, pH 8.5 (125 μl/well). The appropriate antibody coating dilution was determined using standard ELISA checkerboard techniques. The plate was incubated for 2 hours at 37° C., washed 4 times over 10 minutes with Tris buffered saline, containing Tween 20 (TBST) and tapped dry. Standard solutions of tilidine hydrochloride (a mixture of the two trans isomers—the 1R, 2S and the 1S, 2R trans isomers) and nortilidine hydrochloride (a mixture of the two trans isomers—the 1R, 2S and the 1S, 2R trans isomers) were prepared in TBST at 0, 0.625 ng/ml, 1.25 ng/ml, 2.5 ng/ml, 5 ng/ml, 10 ng/ml, 20 ng/ml and 40 ng/ml and 50 μl of each was added to the appropriate wells. 75 μl of conjugate of example 6 (hapten-HRP), diluted in Tris buffer (pH7.2) containing EDTA, D-mannitol, sucrose, thimerosal and BSA was added to each of the wells. The appropriate dilution of conjugate was also determined using standard ELISA checkerboard techniques. The plate was incubated at 25° C. for 1 hour. Excess unbound conjugate was removed by washing 6 times over a 10-15 minute period with TBST. 125 μl of tetramethylbenzidine (TMB) substrate solution was added to each well of the plate that was then incubated for 20 minutes in the dark at room temperature. The reaction was terminated by the addition of 125 μl of 0.2M H₂SO₄ to each well. The absorbance was then measured at 450 nm using a microtitre plate reader.

TABLE 1
Binding characteristics of tilidine-specific antibody raised from Immunogen-II
(example 5) using conjugate of Example 6 as detecting agent

| | Tilidine HCl (a mixture of the two | | Nortilidine HCl (a mixture of the two | |
|---|---|---|---|---|
| | trans isomers - the 1R, 2S and the | | trans isomers - the 1R, 2S and the 1S, | |
| | 1S, 2R trans isomers) | | 2R trans isomers) | |
| Standard ng/ml | A₄₅₀ | % B/Bo | A₄₅₀ | % B/Bo |
| 0 | 1.561 | 100 | 1.551 | 100 |
| 0.625 | 0.989 | 63 | 1.227 | 79 |
| 1.250 | 0.861 | 55 | 1.129 | 73 |
| 2.500 | 0.714 | 46 | 1.004 | 65 |
| 5.000 | 0.572 | 37 | 0.904 | 58 |
| 10.000 | 0.444 | 28 | 0.745 | 48 |
| 20.000 | 0.319 | 20 | 0.571 | 37 |
| 40.000 | 0.212 | 14 | 0.426 | 27 |
| IC₅₀ ng/ml | 1.820 | | 8.522 | |
| % cross-reactivity | 100 | | 21.36 | |

A₄₅₀ = absorbance at 450 nm; B = absorbance at 450 nm at x ng/ml standard concentration; Bo = absorbance at 450 nm at 0 ng/ml standard concentration;
IC₅₀ = standard concentration which produces 50% B/B

### Example 8

### Immunoassay Development

The wells of an enhanced binding 96 well polystyrene microtitre plate were coated with a different Ig fraction (to that exemplified in Example 7) of the antiserum raised to Immunogen II (N-(carboxypropyl)nortilidine-BTG—example 5), diluted in 10 mMTris, pH 8.5 (125 μl/well). The appropriate antibody coating dilution was determined using standard ELISA checkerboard techniques. The plate was incubated for 2 hours at 37° C., washed 4 times over 10 minutes with Tris buffered saline, containing Tween 20 (TBST) and tapped dry. Standard solutions of tilidine hydrochloride (a mixture of the two trans isomers—the 1R, 2S and the 1S, 2R trans isomers) and bisnortilidine hydrochloride (a mixture of the two trans isomers—the 1R, 2S and the 1S, 2R trans isomers) were prepared in TBST at 0, 0.313 ng/ml, 0.625 ng/ml, 1.25 ng/ml, 2.5 ng/ml, 5 ng/ml, 10 ng/ml and 20 ng/ml and 50 μl of each was added to the appropriate wells. 75 μl of conjugate of example 6 (hapten-HRP), diluted in Tris buffer (pH7.2) containing EDTA, D-mannitol, sucrose, thimerosal and BSA was added to each of the wells. The appropriate dilution of conjugate was also determined using standard ELISA checkerboard techniques. The plate was incubated at 25° C. for 1 hour. Excess unbound conjugate was removed by washing 6 times over a 10-15 minute period with TBST. 125 μl of tetramethylbenzidine (TMB) substrate solution was added to each well of the plate that was then incubated for 20 minutes in the dark at room temperature. The reaction was terminated by the addition of 125 μl of 0.2M H₂SO₄ to each well. The absorbance was then measured at 450 nm using a microtitre plate reader.

TABLE 2
Binding characteristics of tilidine-specific antibody raised from Immunogen-II
(example 5) using conjugate of Example 6 as detecting agent

| | Tilidine HCl (a mixture of the tow | | Bisnortilidine HCl (a mixture of the tow | |
|---|---|---|---|---|
| | trans isomers - the 1R, 2S and the | | trans isomers - the 1R, 2S and the 1S, | |
| | 1S, 2R trans isomers) | | 2R trans isomers) | |
| Standard ng/ml | A₄₅₀ | % B/Bo | A₄₅₀ | % B/Bo |
| 0 | 1.382 | 100 | 1.327 | 100 |
| 0.313 | 0.971 | 70 | 1.147 | 86 |
| 0.625 | 0.873 | 63 | 1.083 | 82 |
| 1.250 | 0.724 | 52 | 1.007 | 76 |
| 2.500 | 0.568 | 41 | 0.903 | 68 |
| 5.000 | 0.422 | 31 | 0.786 | 59 |
| 10.000 | 0.297 | 21 | 0.657 | 50 |
| 20.000 | 0.194 | 14 | 0.515 | 39 |

| | | |
|---|---|---|
| IC₅₀ ng/ml | 1.473 | 10.000 |
| % cross-reactivity | 100 | 14.73 |

A₄₅₀ = absorbance at 450 nm; B = absorbance at 450 nm at x ng/ml standard concentration; Bo = absorbance at 450 nm at 0 ng/ml standard concentration;
IC₅₀ = standard concentration which produces 50% B/Bo

Bisnortilidine hydrochloride shows an IC₅₀ of 10.0 ng/ml and a cross-reactivity of 14.73% compared to tilidine hydrochloride (100%).

It is preferred, in a method of detecting or determining tilidine and nortilidine in an in vitro sample of an individual comprising; contacting the sample with the detecting agent of example 6 and the antibody raised from Immunogen-II of example 5, that the IC₅₀ for tilidine hydrochloride is <10 ng/ml, preferably <5 ng/ml, more preferably <2 ng/ml.

Alternatively or additionally, it is preferred, in a method of detecting or determining tilidine and nortilidine in an in vitro sample of an individual comprising; contacting the sample with the detecting agent of example 6 and the antibody raised from Immunogen-II of example 5, that the IC₅₀ for nortilidine hydrochloride is <40 ng/ml, preferably <20 ng/ml, more preferably <10 ng/ml.

Alternatively or additionally, it is preferred, in a method of detecting or determining tilidine and nortilidine in an in vitro sample of an individual comprising; contacting the sample with the detecting agent of example 6 and the antibody raised from Immunogen-II of example 5, that the IC₅₀ for bisnortilidine hydrochloride is <40 ng/ml, preferably <20 ng/ml, more preferably <12.5 ng/ml.

Alternatively or additionally, it is preferred, in a method of detecting or determining tilidine and nortilidine in an in vitro sample of an individual comprising; contacting the sample with the detecting agent of example 6 and the antibody raised from Immunogen-II of example 5, that the cross-reactivity for nortilidine hydrochloride is >10%, preferably >15%, more preferably >20%, compared with 100 for tilidine hydrochloride.

Alternatively or additionally, it is preferred, in a method of detecting or determining tilidine and nortilidine in an in vitro sample of an individual comprising; contacting the sample with the detecting agent of example 6 and the antibody raised from Immunogen-II of example 5, that the cross-reactivity for bisnortilidine hydrochloride is >7.5%, preferably >10%, more preferably >12.5%, compared with 100% for tilidine hydrochloride.

It is preferred, in a kit for detecting or determining tilidine and nortilidine, optionally in an in vitro sample of an individual that, when the sample is contacted with the detecting agent of example 6 and the antibody raised from Immunogen-II of example 5, the IC₅₀ for tilidine hydrochloride is <10 ng/ml, preferably <5 ng/ml, more preferably <2 ng/ml.

Alternatively or additionally, it is preferred, in a kit for detecting or determining tilidine and nortilidine, optionally in an in vitro sample of an individual that, when the sample is contacted with the detecting agent of example 6 and the antibody raised from Immunogen-II of example 5, the IC₅₀ for nortilidine hydrochloride is <40 ng/ml, preferably <20 ng/ml, more preferably <10 ng/ml.

Alternatively or additionally, it is preferred, in a kit for detecting or determining tilidine and nortilidine, optionally in an in vitro sample of an individual that, when the sample is contacted with the detecting agent of example 6 and the antibody raised from Immunogen-II of example 5, the IC₅₀ for bisnortilidine hydrochloride is <40 ng/ml, preferably <20 ng/ml, more preferably <12.5 ng/ml.

Alternatively or additionally, it is preferred, in a kit for detecting or determining tilidine and nortilidine, optionally in an in vitro sample of an individual that, when the sample is contacted with the detecting agent of example 6 and the antibody raised from Immunogen-II of example 5, the cross-reactivity for nortilidine hydrochloride is >10%, preferably >15%, more preferably >20%, compared with 100% for tilidine hydrochloride.

Alternatively or additionally, it is preferred, in a kit for detecting or determining tilidine and nortilidine, optionally in an in vitro sample of an individual that, when the sample is contacted with the detecting agent of example 6 and the antibody raised from Immunogen-II of example 5, the cross-reactivity for bisnortilidine hydrochloride is >7.5%, preferably >10%, more preferably >12.5%, compared with 100 for tilidine hydrochloride.

Alternatively or additionally, the method and the kit of the present invention are useful for detecting or determining bisnortilidine, as well as, tilidine and nortilidine.

Alternatively or additionally, the method and kit of the present invention are useful for detecting or determining, tilidine, nortilidine, salts of tilidine and salts of nortilidine.

Alternatively or additionally, the method and kit of the present invention are useful for detecting or determining, tilidine, demethyl derivatives thereof selected from nortilidine and bisnortilidine, salts of tilidine and the demethyl derivatives of salts of tilidine.

1. A method of detecting or determining tilidine and nortilidine in an in vitro sample of an individual comprising; contacting the sample with one or more detecting agents and one or more antibodies; detecting, or determining the quantity of the one or more detecting agents; and deducing from calibrators the presence of or amount of tilidine and nortilidine in the sample, the one or more antibodies being raised against an immunogen of formula II, 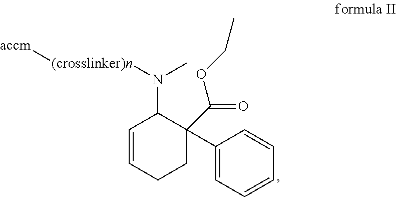
the immunogen comprising a 1S, 2R trans isomer a 1R, 2S trans isomer, or a mixture thereof,
wherein n=0 or 1; and, when n=1, the crosslinker joins the nitrogen atom the antigenicity conferring carrier material (accm).

2. The method of claim 1 for detecting or determining tilidine, nortilidine and bisnortilidine.

3. The method of claim 1, wherein the one or more antibodies are raised against the immunogen of formula II in which the crosslinker is —X—Y—;
wherein, X, which is attached to the nitrogen atom, is selected from the group comprising a substituted or unsubstituted, straight or branched chain, saturated or unsaturated alkylene moiety, or an arylene; and
Y is selected from —C(O)—, —NH—, maleimido, —N—C(O)—, —N—C(S)—, —S—, —S(O)₂—, and —S(O)—.

4. An immunogen of formula II: 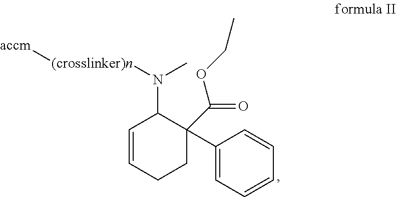
comprising a 1S, 2R trans isomer, a 1R, 2S trans isomer, or a mixture thereof,
wherein n=0 or 1; and, when n=1, the crosslinker joins the nitrogen atom to the antigenicity conferring carrier material (accm).

5. The immunogen of claim 4 in which the crosslinker is —X—Y—;
wherein, X, which is attached to the nitrogen atom, is selected from the group comprising a substituted or unsubstituted, straight or branched chain, saturated or unsaturated alkylene moiety, or an arylene; and
Y is selected from —C(O)—, —NH—, maleimido, —N—C(O)—, —N—C(S)—, —S—, —S(O)₂—, and —S(O)—.

6. An antibody specific to an epitope of tilidine further characterised by being raised against an immunogen of formula II, 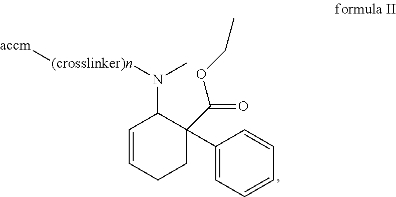
comprising a 1S, 2R trans isomer, a 1R, 2S trans isomer thereof, or a mixture thereof,
wherein n=0 or 1; and, when n=1, the crosslinker joins the nitrogen atom to the antigenicity conferring carrier material (accm).

7. The antibody of claim 6 wherein the antibody has been raised against the immunogen of formula II in which the crosslinker is —X—Y—;
wherein, X, which is attached to the nitrogen atom, is selected from the group comprising a substituted or unsubstituted, straight or branched chain, saturated or unsaturated alkylene moiety, or an arylene; and
Y is selected from —C(O)—, —NH—, maleimido, —N—C(O)—, —N—C(S)—, —S—, —S(O)₂—, and —S(O)—.

8. The antibody of claim 6, wherein the epitope comprises (±)-trans ethyl 2-(amino)-1-phenylcyclohex-3-enylcarboxylate.

9. The antibody of claim 6, wherein the epitope comprises (±)-trans ethyl 2-(amino)-1-phenylcyclohex-3-enylcarboxylate in which the amino is mono or disubstituted with methyl.

10. The antibody of claim 9 which has a cross-reactivity of >10% to nortilidine hydrochloride when compared to 100% for tilidine hydrochloride.

11. The antibody of claim 9 which has a cross-reactivity of >7.5% to bisnortilidine hydrochloride when compared to 100% for tilidine hydrochloride.

12. A kit for detecting or determining tilidine and nortilidine, the kit comprising at least one antibody of claim 6.

13. The kit of claim 12 comprising said at least one antibody for detecting or determining tilidine, nortilidine and bisnortilidine.

14. The method of claim 1, wherein the immunogen comprises a racemic mixture of the trans 1S, 2R and trans 1R, 2S isomers.

15. The immunogen of claim 4, wherein the immunogen comprises a racemic mixture of the trans 1S, 2R and trans 1R, 2S isomers.

16. The antibody of claim 6, wherein the immunogen comprises a racemic mixture of the trans 1S, 2R and trans 1R, 2S isomers.
